(19) United States
(12) Patent Application Publication
(10) Pub. No.: US 2023/0086471 A1
(43) Pub. Date: Mar. 23, 2023

(54) A METHOD TO AMPLIFY A NUCLEIC ACID

(71) Applicant: Agency for Science, Technology and Research, Singapore (SG)

(72) Inventors: Jackie Y. Ying, Singapore (SG); Muhammad Nadjad Bin Abdul Rahim, Singapore (SG)

(73) Assignee: Agency for Science, Technology and Research, Singapore (SG)

(21) Appl. No.: 17/799,882

(22) PCT Filed: Feb. 15, 2021
(86) PCT No.: PCT/SG2021/050075

§ 371 Date: Aug. 15, 2022

(30) Foreign Application Priority Data
Feb. 15, 2020 (SG) 10202001393W

(51) Int. Cl. C12Q 1/6844

(52) U.S. Cl. CPC C12Q 1/6844

(57) ABSTRACT

This invention relates to methods and compositions for amplifying nucleic acids, e.g., genomic DNA, using nicking agents. The method of amplifying nucleic acids comprising: (a) forming a reaction mixture comprising: (i) a first nucleic acid template comprising a strand having a first nicking agent recognition sequence; (ii) a second nucleic acid template comprising a strand having a second nicking agent recognition sequence; (iii) at least one primer for a target region on the first or second nucleic acid template; (iv) at least one protein having DNA polymerase domain function, wherein the domain function comprises a first domain function capable of strand displacement activity and a second domain function capable of high processivity activity, or at least one protein having DNA polymerase domain function capable of strand displacement activity and at least one protein having DNA polymerase domain function capable of high processivity activity; (v) at least one deoxynucleoside triphosphate; and (vi) a first nicking agent for recognizing the first nicking agent recognition sequence and a second nicking agent for recognizing the second nicking agent recognition sequence; (b) incubating the reaction mixture under conditions that amplifies the nucleic acid templates, wherein the domain functions capable of strand displacement activity and high processivity activity are separate from each other and capable of carrying out their activities simultaneously. In specific embodiments, the nicking agent is NB.BsrDI and the proteins having DNA polymerase domain functions are Bst 3.0 polymerase and Pfu polymerase.

This invention relates to the field of molecular biology, more particularly to methods and compositions involving nucleic acids, and still more particularly to methods and compositions for amplifying nucleic acids, e.g., genomic DNA, using nicking agents.

A number of methods have been developed for whole genome amplification. Most of these methods involve the use of random or partially random primers to amplify the entire genome of an organism in a PCR reaction (see, e.g., Kuukasjarvi et al., Genes, Chromosomes and Cancer 18: 94-101 (1997); Telenius et al., Genomics 13: 718-25, 1992; Zhang et al., Proc. Natl. Acad. Sc. USA 89: 5847-51, 1992; Cheung et al., Proc. Natl. Acad. Sci. 93: 14676-79, 1996; Barrett et al., Nucleic Acids Res. 23: 3488-92; Klein et al., Proc. Natl. Acad. Sci. USA 96: 4494-9, 1999; Sun et al., Nucleic Acids Res. 23: 3034-40, 1995; Larsen et al., Cytometry 44: 317-325, 2001; and Barbaux et al., J. Mol. Med. 79: 329-32, 2001). This technique relies on having a sufficient number of primers of random or partially random sequences so that pairs of primers hybridize throughout the genomic DNA at moderate intervals. Extension from the 3′ termini of the primers produces strands to which another primer anneals. By subjecting the genomic DNA to multiple amplification cycles, the genomic sequences are amplified. Since this technique relies on PCR, it has the disadvantage that the amplification reaction must be carried out under cycles of different temperatures to achieve cycles of denaturation and re-annealing. Such cycles of denaturation and re-annealing are disadvantageous for many reasons, e.g., they may cause gene shuffling artifacts.

In addition, while the PCR has made tremendous impact and has led to significant developments in healthcare, biotechnology, education, history, archaeology, information technology, safety and security, it requires the use of significant infrastructure and takes usually at least 1-2 hours to complete a run.

An alternative method for whole genome amplification is known as whole genome strand displacement amplification. This technique involves hybridization of random or partially random primers to a target genomic DNA and replication of the target sequence primed by the hybridized primers so that replication of the target sequence results in replicated strands complementary to the target sequence (see, e.g., U.S. Pat. Nos. 6,124,120 and 6,280,949). During replication, the growing replicated strands displace other replicated strands from the target sequence (or from another replicated strand) via strand displacement replication. Displacement of replicated strands by other replicated strands allows the amplification of a target sequence or portions thereof. Such isothermal amplification methods markedly reduce the infrastructure and can be faster than PCR, and some isothermal amplification methods can lead to significant decrease in time-to-result. However, such isothermal techniques may be slow and requires the use of multiple primers.

As such, there is a need in the art for a simpler and more efficient and quicker method to amplify nucleic acids.

The listing or discussion of an apparently prior-published document in this specification should not necessarily be taken as an acknowledgement that the document is part of the state of the art or is common general knowledge.

Any document referred to herein is hereby incorporated by reference in its entirety.

In an aspect of the invention, there is provided a method to amplify nucleic acids, the method comprising:
(a) forming a reaction mixture comprising:
   (i) a first nucleic acid template comprising a strand having a first nicking agent recognition sequence;
   (ii) a second nucleic acid template comprising a strand having a second nicking agent recognition sequence;
   (iii) at least one primer for a target region on the first or second nucleic acid template;
   (iv) at least one protein having DNA polymerase domain function, wherein the domain function comprises a first domain function capable of strand displacement activity and a second domain function capable of high processivity activity, or at least one protein having DNA polymerase domain function capable of strand displacement activity and at least one protein having DNA polymerase domain function capable of high processivity activity;
   (v) at least one deoxynucleoside triphosphate; and
   (vi) a first nicking agent for recognizing the first nicking agent recognition sequence and a second nicking agent for recognizing the second nicking agent recognition sequence;
(b) incubating the reaction mixture under conditions that amplifies the nucleic acid templates,
wherein the domain functions capable of strand displacement activity and high processivity activity are separate from each other and capable of carrying out their activities simultaneously.

By “nucleic acid”, it is meant to include any polymer of nucleotides and includes DNA or RNA. By “nucleic acid template”, it is meant to region to that region of interest for amplification. Here, the nucleic acid template may be single or double stranded. The terms ″3″′ and ″5″′ are used herein to describe the location of a particular site within a single strand of nucleic acid. When a location in a nucleic acid is “3′ to″ or ″3′ of” a nucleotide reference or string of nucleotides, this means that the location is between the reference nucleotide(s) and the 3 hydroxyl of that strand of nucleic acid. Likewise, when a location in a nucleic acid is “5′ to″ or ″5′ of” a reference nucleotide, this means that it is between the reference nucleotide and the 5′ phosphate of that strand of nucleic acid.

By “amplify”, it is meant to include the making of one, two, three or more copies of a nucleic acid molecule (either single-stranded, e.g., produced via strand displacement amplification; or double-stranded, e.g., produced via polymerase chain reaction) by a DNA polymerase using one strand, both strands of a double-stranded target nucleic acid molecule (or multiple target nucleic acid molecules with identical sequences), or a portion of one strand or both strands as a template (or templates). The newly made nucleic acid molecules should comprise a nucleotide sequence identical to at least a portion of the target or template nucleic acid.

As such, “nucleic acid template” includes any “target polynucleotide template” or “template” that refers to a polynucleotide containing an amplified region. The “amplified region,” is a region of a polynucleotide that is to be, for example, synthesized by polymerase chain, reaction (PCR).

The templates that are to be amplified by a method of the invention may be single or double stranded. As such, the invention includes amplification of at least a portion of a double-stranded target nucleic acid refers to the making of one, two, three or more copies of such a nucleic acid molecule.

By “nicking agent recognition sequence”, it is meant to refer to a sequence which is recognized by a nicking agent. The term “nicking”, as used herein, refers to the cleavage of only one strand of the double-stranded portion of a fully or partially double-stranded nucleic acid. The position where the nucleic acid is nicked is referred to as the nicking site, which be or part of the nicking agent recognition sequence. A “nicking agent” is an agent that nicks a partially or fully double-stranded nucleic acid. It may be an enzyme or any other chemical compound or composition. In certain embodiments, a nicking agent may recognize a particular nucleotide sequence of a fully or partially double-stranded nucleic acid and cleaves only one strand of the fully or partially double-stranded nucleic acid at a specific position (i.e., the nicking site) relative to the location of the recognition sequence. Such nicking agents (referred to as “specific nicking agents” include, but are not limited to, a nicking endonuclease (e.g., N.BstNB I), and a restriction endonuclease (e.g., Hinc II) when the fully or partially double-stranded DNA contains a hemimodified recognition/cleavage site in which one strand contains at least one derivatized nucleotide that prevents cleavage of one strand (i.e., the strand that contains the derivatized nucleotide or the other strand that does not contain the derivatized nucleotide) by the restriction endonuclease. In some embodiments, a nicking agent may be an agent that does not require a specific recognition sequence in a double-stranded target nucleic acid and creates one or more randomly placed nicks in the target. Such a nicking agent is referred to as a random nicking agent and may be an enzyme or any other chemical compound or composition.

In various embodiments, a nicking agent recognition sequence is a double-stranded nucleotide sequence where each nucleotide in one strand of the sequence is complementary to the nucleotide at its corresponding position in the other strand. In such embodiments, the sequence of a nicking agent recognition sequence in the strand containing a nicking site nickable by a nicking agent that recognizes the nicking agent recognition sequence is referred to as a “sequence of the sense strand of the nicking agent recognition sequence” or a “sequence of the sense strand of the double-stranded nicking agent recognition sequence,” while the sequence of the nicking agent recognition sequence in the strand that does not contain the nicking site is referred to as a “sequence of the antisense strand of the nicking agent recognition sequence” or a “sequence of the antisense strand of the double-stranded nicking agent recognition sequence.”

In various embodiments, a nicking agent recognition sequence is an at most partially double-stranded nucleotide sequence that has one or more nucleotide mismatches, but contains an intact sense strand of a double-stranded nicking agent recognition sequence as described above. According to the convention used herein, in the context of describing a nicking agent recognition sequence, when two nucleic acid molecules anneal to one another so as to form a hybridized product, and the hybridized product includes a nicking agent recognition sequence, and there is at least one mismatched base pair within the nicking agent recognition sequence of the hybridized product, then this nicking agent recognition sequence is considered to be only partially double-stranded. Such nicking agent recognition sequences may be recognized by certain nicking agents that require only one strand of double-stranded recognition sequences for their nicking activities.

In various embodiments, a nicking agent recognition sequence is a partially or completely single-stranded nucleotide sequence that has one or more unmatched nucleotides, but contains an intact sense strand of a double-stranded nicking agent recognition sequence as described above. According to the convention used herein, in the context of describing a nicking agent recognition sequence, when two nucleic acid molecules (i.e., a first and a second strand) anneal to one another so as to form a hybridized product, and the hybridized product includes a nucleotide sequence in the first strand that is recognized by a nicking agent, i.e., the hybridized product contains a nicking agent recognition sequence, and at least one nucleotide in the sequence recognized by the nicking agent does not correspond to, i.e., is not across from, a nucleotide in the second strand when the hybridized product is formed, then there is at least one unmatched nucleotide within the nicking agent recognition sequence of the hybridized product, and this nicking agent recognition sequence is considered to be partially or completely single-stranded. Such nicking agent recognition sequences may be recognized by certain nicking agents that require only one strand of double-stranded recognition sequences for their nicking activities.

In various embodiments, the first and second nicking agent recognition sequences are the sense strands of the respective nucleotide sequence. In alternative embodiments, the first and second nicking agent recognition sequences are the antisense strands of the respective nucleotide sequence.

By “primer”, it is meant to refer to any suitable single stranded DNA or RNA molecule that can hybridize to a polynucleotide or nucleic acid template and prime enzymatic synthesis of a second polynucleotide strand. A primer useful according to the invention may be between 10 to 100 nucleotides in length, preferably 17-50 nucleotides in length and more preferably 17-45 nucleotides in length.

As used herein, “polymerase” refers to an enzyme that catalyzes the polymerization of nucleotide (i.e., the polymerase activity). Generally, the enzyme will initiate synthesis at the 3′-end of the primer annealed to a polynucleotide template sequence, and will proceed toward the 5′ end of the template strand. A “DNA polymerase” catalyzes the polymerization of deoxynucleotides. The DNA polymerase may be thermostable. The DNA polymerase may be an archaeal DNA polymerase.

By “DNA polymerase domain function”, it is meant to refer to the activity of a DNA polymerase, described herein. Activities of the DNA polymerase include, but are not limited to, processivity, salt-resistance, DNA binding, strand displacement activity, polymerase activity, nucleotide binding and recognition, 3′-5′ or 5′→3′ exonuclease activities, proofreading, fidelity and/or decreased DNA polymerization at room temperature, as defined hereinbelow. DNA polymerase activities are well known in the art (Ausubel et. al. Short Protocols in Molecular Biology (1995) 3rd Ed. John Wiley & Sons, Inc.; (Sambrook et al., (1989) in: Molecular Cloning, A Laboratory Manual (2nd Ed.), Cold Spring Harbor Laboratory Press, Cold Spring Harbor, N.Y., see additional references incorporated by reference in their entirety herein); Stratagene Catalog).

By “domain”, it is meant to refer to a unit of a protein or protein complex, comprising a polypeptide subsequence, a complete polypeptide sequence, or a plurality of peptide sequences. A “domain” useful according to the invention includes any double stranded or single stranded DNA binding domain known in the art or that becomes known in the art.

Here, the DNA polymerase “function” also includes an activity of a “mutant” DNA polymerase, as defined herein. The invention encompasses but is not limited to the following activities of a “mutant” according to the invention: base analog detection activities, DNA polymerization activity, reverse transcriptase activity, processivity, salt resistance, DNA binding, strand displacement activity, nucleotide binding and recognition, 3′-5′ or 5′→3′ exonuclease activities, proofreading, fidelity, efficiency, specificity, thermostability and intrinsic hot start capability or decreased DNA polymerization at room temperature, decreased amplification slippage on templates with tri-nucleotide repeat stretches, decreased amplification cycles, decreased extension times, and a decrease in the amount of polymerase needed for the applications described herein. In one embodiment, the “mutant” polymerase of the invention refers to a DNA polymerase containing one or more mutations that reduce one or more base analog detection activities of the DNA polymerase. In one embodiment, a “mutant” refers to a polymerase that has a mutation that confers an improved polymerization rate or fidelity on the polymerase. In a preferred embodiment, the “mutant” polymerase of the invention has a reduced uracil detection activity. In a preferred embodiment, the “mutant” polymerase of the invention has a reduced inosine detection activity. In another preferred embodiment, the “mutant” polymerase of the invention has a reduced uracil and inosine detection activity. In another preferred embodiment, the “mutant” polymerase of the invention has a reduced DNA polymerization activity. Any of the “mutants”, for example, a mutant with reduced uracil activity, may also possess improved polymerization rate and/or fidelity, as compared to a wild-type polymerase.

In various embodiments, the protein having DNA polymerase domain function comprises a first domain capable of strand displacement activity and a second domain capable of high processivity activity, the first and second domains are separate from each other and capable of carrying out their activities simultaneously.

As such, the protein having DNA polymerase domain function may be a result of a fusion.

By “fusion”, it is meant to include any protein that is “fused” or “joined” by any method known in the art for functionally connecting polypeptide domains, including without limitation recombinant fusion with or without intervening domains, intein-mediated fusion, non-covalent association, and covalent bonding, including disulfide bonding, hydrogen bonding, electrostatic bonding, and conformational bonding.

A “fusion” may include any protein where a first amino acid sequence (protein) comprising a wild type or mutant DNA polymerase of the invention, joined to a second amino acid sequence defining a polypeptide that modulates one or more activities of the DNA polymerase including, but not limited to, processivity, salt-resistance, DNA binding, strand displacement activity, polymerase activity, nucleotide binding and recognition, 3′-5′ or 5′-3′ exonuclease activities, proofreading, fidelity and/or decreased DNA polymerization at room temperature, wherein the first and second amino acids are not found in the same relationship in nature. A “fusion” may include two or more amino acid sequences (for example a sequence encoding a wild type or mutant DNA polymerase and a polypeptide that increases processivity and/or salt resistance) from unrelated proteins, joined to form a new functional protein.

In addition, or as an alternative, to a “fusion” protein, the protein having DNA polymerase domain function may be a “mutant” protein as indicated above. Generally, “mutation” refers to a change introduced into a parental or wild type DNA sequence that changes the amino acid sequence encoded by the DNA, including, but not limited to, substitutions, insertions, deletions or truncations. The consequences of a mutation include, but are not limited to, the creation of a new character, property, function, or trait not found in the protein encoded by the parental DNA, including, but not limited to, N terminal truncation, C terminal truncation or chemical modification.

These mutations modulate one or more activities of the protein including, but not limited to, base analog detection activities, DNA polymerization activity, reverse transcriptase activity, processivity, salt resistance, DNA binding, strand displacement activity, nucleotide binding and recognition, 3′-5′ or 5′→3′ exonuclease activities, proofreading, fidelity, efficiency, specificity, thermostability and intrinsic hot start capability or decreased DNA polymerization at room temperature, decreased amplification slippage on templates with tri-nucleotide repeat stretches, decreased amplification cycles, decreased extension times, and a decrease in the amount of polymerase needed for the applications described herein. In various embodiments, the “mutant” polymerase of the invention may be a DNA polymerase containing one or more mutations that reduce one or more base analog detection activities of the DNA polymerase. In various embodiments, a “mutant” refers to a polymerase that has a mutation that confers an improved polymerization rate or fidelity on the polymerase. In various embodiments, the “mutant” polymerase of the invention has a reduced uracil detection activity. In various embodiments, the “mutant” polymerase of the invention has a reduced inosine detection activity. In various embodiments, the “mutant” polymerase of the invention has a reduced uracil and inosine detection activity.

In various embodiments, the “mutant” polymerase of the invention has a reduced DNA polymerization activity. Any of the “mutants” for example a mutant with reduced uracil activity, may also possess improved polymerization rate and/or fidelity, as compared to a wild-type polymerase. A “mutant” polymerase as defined herein, includes a polymerase comprising one or more amino acid substitutions, one or more amino acid insertions, a truncation or an internal deletion. A “mutant” polymerase as defined herein includes non-fusion and fusion polymerases.

In addition, the “mutant” polymerase may include a fusion polymerase (as described above) wherein any of the single, double or triple mutant DNA polymerases described herein, any mutant DNA polymerase comprising an insertion, described herein, or any of the truncated, or deleted mutant DNA polymerases described herein, occur in combination with a polypeptide that modulates one or more activities of the DNA polymerase including, but not limited to, DNA polymerization activity, base analog detection activities, DNA polymerization activity, reverse transcriptase activity, processivity, salt resistance, DNA binding, strand displacement activity, nucleotide or nucleotide analog binding and recognition, sensitivity to uracil, 3′-5′ or 5′→3′ exonuclease activities, proofreading, fidelity efficiency, specificity, thermostability and intrinsic hot start capability or decreased DNA polymerization at room temperature, decreased amplification slippage on templates with tri-nucleotide repeat stretches, decreased amplification cycles, decreased extension times, and a decrease in the amount of polymerase needed for the applications described herein, thereby forming a fusion, as defined herein. For example, a polypeptide that increases processivity and or salt resistance is described in WO 01/92501 A1 and Pavlov et al., 2002, Proc. Natl. Acad. Sci. USA, 99:13510-13515, herein incorporated by reference in their entirety.

In various embodiments of the invention, the protein having DNA polymerase domain function of the invention (be it “fusion” or “mutant” or otherwise) comprises two domains capable of both activities, i.e. strand displacement activity and a second domain capable of high processivity activity.

By “strand displacement activity”, it is meant to refer to an activity of the protein having DNA polymerase domain function that can synthesize DNA by unwinding template without a helicase activity. The term “synthesize” includes any in vitro method for making a new strand of polynucleotide or elongating existing polynucleotide (i.e., DNA or RNA) in a template dependent manner, using any desired template to be amplified. Synthesis, according to the invention, includes amplification (as defined above), which increases the number of copies of a polynucleotide template sequence with the use of a polymerase. Polynucleotide synthesis (e.g., amplification) results in the incorporation of nucleotides into a polynucleotide (i.e., a primer), thereby forming a new polynucleotide molecule complementary to the polynucleotide template. “Complementary” refers to the broad concept of sequence complementarity between regions of two polynucleotide strands or between two nucleotides through base-pairing. It is known that an adenine nucleotide is capable of forming specific hydrogen bonds (“base pairing”) with a nucleotide which is thymine or uracil. Similarly, it is known that a cytosine nucleotide is capable of base pairing with a guanine nucleotide.

In various embodiments of the invention, the amplification method requires the use of at least one protein having DNA polymerase domain function capable of strand displacement activity and at least one protein having DNA polymerase domain function capable of high processivity activity. In other words, two distinct and separate DNA polymerases may be used. For example, one DNA polymerase exhibits or is capable of strand displacement activity only, while the other DNA polymerase exhibits or is capable of high processivity activity only.

The nicking of the target nucleic acid produces 3′ termini at the nicking sites, from which extension may be performed in the presence of a DNA polymerase. When the DNA polymerase lacks a 5′→3′ exonuclease activity, but has a strand displacement activity, the extension of the nicked template nucleic acid at the nicking site displaces the downstream single-stranded nucleic acid fragment. Such displacement allows the accumulation, thus amplification, of the single-stranded nucleic acid fragment.

Any DNA polymerase that is 5′→3′ exonuclease deficient but has a strand displacement activity may be used to extend from a nicked template nucleic acid and to subsequently amplify a single-stranded nucleic acid in the continuous presence of a nicking agent. Such DNA polymerases include, but are not limited to, exo− Deep Vent, exo− Bst, exo− Pfu, and exo− Bca. Additional DNA polymerase useful in the present invention may be screened for or created by the methods described in U.S. Pat. No. 5,631,147, incorporated herein by reference in its entirety. The strand displacement activity may be further enhanced by the presence of a strand displacement facilitator as described below.

A DNA polymerase that does not have a strand displacement activity may be used. Such DNA polymerases include, but are not limited to, exo− Vent, Taq, the Klenow fragment of DNA polymerase I, T5 DNA polymerase, and Phi29 DNA polymerase. In certain embodiments, the use of these DNA polymerases requires the presence of a strand displacement facilitator. A “strand displacement facilitator” is any compound or composition that facilitates strand displacement during nucleic acid extensions from a 3′ terminus at a nicking site catalyzed by a DNA polymerase. Exemplary strand displacement facilitators useful in the present invention include, but are not limited to, BMRF1 polymerase accessory subunit (Tsurumi et al., J. Virology 67: 7648-53, 1993), adenovirus DNA-binding protein (Zijderveld and van der Vliet, J. Virology 68: 1158-64, 1994), herpes simplex viral protein ICP8 (Boehmer and Lehman, J. Virology 67: 711-5, 1993; Skaliter and Lehman, Proc. Natl. Acad. Sci. USA 91: 10665-9, 1994), single-stranded DNA binding protein (Rigler and Romano, J. Biol. Chem. 270: 8910-9, 1995), phage T4 gene 32 protein (Villemain and Giedroc, Biochemistry 35: 14395-4404, 1996), calf thymus helicase (Siegel et al., J. Biol. Chem. 267: 13629-35, 1992) and trehalose. In one embodiment, trehalose is present in the amplification reaction mixture.

Additional exemplary DNA polymerases useful in the present invention include, but are not limited to, phage M2 DNA polymerase (Matsumoto et al., Gene 84: 247, 1989), phage PhiPRD1 DNA polymerase (Jung et al., Proc. Natl. Acad. Sci. USA 84: 8287, 1987), T5 DNA polymerase (Chatterjee et al., Gene 97: 13-19, 1991), Sequenase (U.S. Biochemicals), PRD1 DNA polymerase (Zhu and Ito, Biochim. Biophys. Acta. 1219: 267-76, 1994), 9° Nm™ DNA polymerase (New England Biolabs) (Southworth et al., Proc. Natl. Acad. Sci. 93: 5281-5, 1996; Rodriquez et al., J. Mol. Biol. 302: 447-62, 2000), and T4 DNA polymerase holoenzyme (Kaboord and Benkovic, Curr. Biol. 5: 149-57, 1995).

Alternatively, a DNA polymerase that has a 5′→3′ exonuclease activity may be used. For instance, such a DNA polymerase may be useful for amplifying short nucleic acid fragments that automatically dissociate from the template nucleic acid after nicking.

The formed polynucleotide molecule and its template can be used as templates to synthesize additional polynucleotide molecules. “DNA synthesis,” as used herein, includes, but is not limited to, PCR, the labeling of polynucleotide (i.e., for probes and oligonucleotide primers), polynucleotide sequencing.

By “primer”, it is mean to refer to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and polymerase in an appropriate buffer (“buffer” includes pH, ionic strength, cofactors, etc.) and at a suitable temperature. The primer is preferably single-stranded for maximum efficiency in amplification, but may alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the polymerase. The exact lengths of the primers will depend on many factors, including temperature, source of primer and use of the method. For example, depending on the complexity of the target sequence, the oligonucleotide primer typically contains 15-25 nucleotides, although it may contain more or few nucleotides. Short primer molecules generally require colder temperatures to form sufficiently stable hybrid complexes with template.

By “processivity activity”, it is meant to refer to the ability of the protein having DNA polymerase domain function to remain attached to the template or substrate and perform multiple modification reactions. “Modification reactions” include but are not limited to polymerization, and exonucleolytic cleavage. “Processivity” also refers to the ability of a nucleic acid modifying enzyme, for example a polymerase, to modify relatively long (for example 0.5-1 kb, 1-5 kb or 5 kb or more) tracts of nucleotides. “Processivity” also refers to the ability of a nucleic, acid modifying enzyme, for example a DNA polymerase, to perform a sequence of polymerization steps without intervening dissociation of the enzyme from the growing DNA chains. “Processivity” can depend on the nature of the polymerase, the sequence of a DNA template, and reaction conditions, for example, salt concentration, temperature or the presence of specific proteins.

As used herein, “high processivity” or “increased processivity” refers to an increase of 5-10%, preferably 10-50%, more preferably 50-100% or more, as compared to a wild type or mutant archael DNA polymerase that lacks a polypeptide that increases processivity and/or salt resistance as defined herein. Processivity and increased processivity can be measured according to the methods defined herein and in Pavlov et al., supra and WO 01/92501 A1. A polymerase with increased processivity that is a chimera comprising a polypeptide that increases processivity, as defined herein, is described in Pavlov et al. supra and WO 01/92501 A1.

In particular, the term “high processivity” refers to a processivity higher than 20 nts (e.g., higher than 40 nts, 60 nts, 80 nts, 100 nts, 120 nts, 140 nts, 160 nts, 180 nts, 200 nts, 220 nts, 240 nts, 260 nts, 280 nts, 300 nts, 320 nts, 340 nts, 360 nts, 380 nts, 400 nts, or higher) per association/disassociation with the template. This would be equivalent to a “high elongation rate” which may refer to an elongation rate higher than 25 nt/s (e.g., higher than 30, 35, 40, 45, 50, 55, 60, 65, 70, 75, 80, 85, 90, 95, 100, 105, 110, 115, 120, 125, 130, 135, 140 nt/s). In various embodiments, “high processivity” refers to at least 24 base pairs of the nucleic being processed/copied by the protein having DNA polymerase domain function.

The second domain capable of high processivity activity may also be or include a “processivity factor” which is meant to include any protein or polypeptide which is able to increase the processivity activity of protein having DNA polymerase domain activity by at least two and preferably 20, 50 or more fold. Such a processivity factor may be part of the protein or binds to the protein and thereby increases the processivity of that protein, i.e. the protein having DNA polymerase domain activity. A “binding domain” may refer to that portion of the protein (DNA polymerase) which is involved in binding of a processivity factor, as can be measured by standard procedures.

In various embodiments, the first and second domains are separated by a linker to form a chimeric protein. Any suitable linkers may be used, e.g. an amino acid chain, peptides etc.

By “chimeric”, it is meant that the protein having DNA polymerase domain function includes one or several amino acids (not present in a corresponding wild-type polymerase) which either modify an existing strand displacement activity domain and/or processivity activity domain, or introduce a strand displacement activity and/or new processivity domain into the protein having DNA polymerase domain function. Generally, such a chimeric DNA polymerase will have inserted therein at least 20 or more amino acids, preferably at least 50 or even 100 amino acids in a corresponding naturally occurring DNA polymerase.

In various embodiments, such a chimeric DNA polymerase is a man-made object and is not found in nature as a wild-type polymerase. The region to be inserted as described herein is usually not naturally-occurring in the enzyme in which it is inserted but is taken from an enzyme in which it naturally occurs.

In various embodiments, the protein having DNA polymerase domain function may be exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9°Nm™ DNA polymerase, T4 DNA polymerase, strand displacing Taq polymerase, or combinations thereof.

In addition to a protein having DNA polymerase domain function, the reaction mixture of the invention may include any suitable reverse transcriptase.

The methods of nucleic acid amplification of the present invention employs exponential amplification techniques, i.e. the target templates generate amplicons which later gets reamplified. These techniques are set out in WO2004067726. As such, in various embodiments, the second template further comprises a region that is not substantially complementary to the at least one primer, and incubating the reaction mixture under conditions to amplify (a) a first single-stranded nucleic acid molecule using the first template nucleic acid as a template; and (b) a second single-stranded nucleic acid molecule using the second template nucleic acid as a template.

In various embodiments, the reaction mixture further comprises a third nucleic acid template comprising: (a) a third nicking agent recognition sequence; (b) a first region that is substantially identical or complementary to the at least one primer; and (c) a second region that is not substantially identical or complementary to the at least one primer.

In various embodiments, the third nicking agent recognition sequence is an antisense sequence strand.

In various embodiments, the first, second and third nicking agent recognition sequences are identical.

In various embodiments, the nicking agent is a nicking endonuclease selected from the group consisting of Nb.BbvCI, Nb.Bsml, Nb.BsrDI, Nb.BssSI, Nb.Btsl, Nt.Alwl, Nt.BbvCI, Nt.BsmAl, Nt.BspQI, Nt.BstNBl, Nt.CviPII, Zinc Finger nickase, TALE nickase, Cas nickase, and combinations thereof.

A “nicking endonuclease”, may be used and is meant to refer to an endonuclease that recognizes a nucleotide sequence of a completely or partially double-stranded nucleic acid molecule and cleaves only one strand of the nucleic acid molecule at a specific location relative to the recognition sequence. Unlike a restriction endonuclease, which requires its recognition sequence to be modified by containing at least one derivatized nucleotide to prevent cleavage of the derivatized nucleotide-containing strand of a fully or partially double-stranded nucleic acid molecule, a nicking endonuclease typically recognizes a nucleotide sequence composed of only native nucleotides and cleaves only one strand of a fully or partially double-stranded nucleic acid molecule that contains the nucleotide sequence.

In various embodiments, the 3′ terminus of the first nucleic acid template or the second nucleic acid template is blocked.

Suitable 3′ blocking groups and methods for removing the 3′ blocking groups include, but are not limited to, the 3′ blocking groups and methods described in U.S. Pat. No. 7,541,444, which is incorporated by reference herein in its entirety. By way of example, suitable 3′ blocking groups include, but are not limited to, 3′ddC, 3′ Inverted dT, 3′ C3 spacer, 3′ Amino, and 3′ phosphorylation. They can also include PEG linker groups on the 3′ ends.

In various embodiments, the 3′-end may be blocked with, for example, a phosphate, an amine, a biotin, a dideoxy group or a fluorophore (that is, there is no free 3′-hydroxyl in T2) to prevent extension. The 3′ ends of the templates may be blocked to prevent them acting as primers through mis-pairing with each other.

In various embodiments, the first nucleic acid template and the second nucleic acid template are immobilized. may be immobilized in different regions of a solid substrate or different solid substrates (e.g., microbeads).

In various embodiments, the first nucleic acid template or the second nucleic acid template comprises nucleic acid modifications. There are numerous modifications that can be done to the nucleic acid, for example unnatural bases, locked nucleic acids etc. In an example, these templates may be chemically modified. By “chemically modified”, it is mean to refer to a nucleic acid that may be chemically or biochemically modified or contains non-natural or derivatized nucleotide bases. Such modifications include, for example, labels, methylation, substitution of one or more of the naturally occurring nucleotides with an analog, internucleotide modifications such as uncharged linkages (e.g. methyl phosphonates, phosphorodithioates, etc.), pendent moieties (e.g., polypeptides), intercalators, (e.g. acridine, psoralen, etc.) chelators, alkylators, and modified linkages (e.g. alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In various embodiments, the first nucleic acid template or the second nucleic acid template is about 6 to 20,000 nucleotides in length.

In various embodiments, the method further comprises the step of detecting or characterizing the amplified nucleic acid(s). Any suitable detection methods may be performed, such as luminescence spectroscopy or spectrometry, fluorescence spectroscopy or spectrometry, mass spectrometry, liquid chromatography, fluorescence polarization, electrophoresis, mass spectrometry, SYBR I fluorescence, SYBR II fluorescence, SYBR Gold, Pico Green, Evagreen, TOTO-3, intercalating dye detection, FRET, molecular beacon detection, scorpion probe detection, surface capture, capillary electrophoresis, incorporation of labeled nucleotides to allow detection by capture, fluorescence polarization, lateral flow capture, and combinations thereof.

In various embodiments, amplification occurs in the presence of a probe. The probe can be, for example, a fluorescent reporter probe. In various embodiments, the amplification occurs in the presence of a nucleic acid binding agent. Nucleic acid binding agents, include, but are not limited to, intercalating agents, major and minor nucleic acid groove binders and nucleic acid stains. Such agents are known and commercially available, e.g., from Molecular Probes, Inc. (Eugene, Oreg.). Optionally, the nucleic acid binding agent is selected from the group consisting of SYBR green, SYBR blue, DAPI, propidium iodine, Hoeste, SYBR gold, and ethidium bromide.

In various embodiments, the reaction mixture further comprises a template conjugate for linking the first and second nucleic acid templates. In various embodiments, the 3′ terminus of the first oligonucleotide template is linked to the 5′ terminus of the second oligonucleotide template via the conjugate. In various embodiments, the 3′ terminus of the first oligonucleotide template is linked to the 3′ terminus of the second oligonucleotide template via the conjugate.

The templates can be bound to each other using simple cross-linking chemistries such as 3′-amines or 5′ amines (or both) and a crosslinker like cyanuric chloride.

The template of the invention may be a multimer formed by linking multiple molecules of a single template nucleic acid (e.g., A-linker-A-linker-A-linker-A-linker-A-linker-A) or by linking multiple molecules of different template nucleic acids (e.g., A-linker-B-linker-A-linker-B-linker-A-linker-B). The multimers can be linear or circular. The minimum number of individual template nucleic acid molecules to be cross-linked are 2, the maximum number is 1 billion. Preferably, 2 to 400 individual template molecules are to linked together. Also preferably, the template nucleic acids are linked together at the 3′ end of their individual components to prevent self- or mis- priming reactions. Linking individual template oligonucleotides together may also increase the rates of reactions. The above multimers (also referred to as “oligonucleotide conjugates”) can be easily prepared and purified by size exclusion chromatography or HPLC.

The templates can also be cross-linked onto dendrimer polymers, or polymers like polylysine, poly(ethyleneimine). The template oligonucleotides can be bound to the polymers using simple cross-linking chemistries such as 3′-amines or 5′ amines (or both) and a crosslinker like cyanuric chloride. Oligonucleotides-polymer conjugates can be easily prepared and purified by size exclusion chromatography. The variations described above may be combined with each other or with the linear amplification or exponential amplification schemes.

In various embodiments, the first nucleic acid template is identical to the second nucleic acid template.

In various embodiments, the first nicking agent recognition sequence is identical to the second nicking agent recognition sequence.

In various embodiments, the nucleic acid template further comprises a detectable label. In an example, the detectable label is a fluorescent moiety. Detectable labels include any substance which is capable of producing a signal that is detectable by visual or instrumental means. Suitable labels include, but are not limited to, labels which produce signals through either chemical or physical means, such as fluorescent dyes, chromophores, electrochemical moieties, enzymes, radioactive moieties, phosphorescent groups, fluorescent moieties, chemiluminescent moieties, or quantum dots.

In various embodiments, the amplification has the kinetic that fits the equation σ˜σ₀e^{βt}, where σ₀ is an initial concentration of the oligonucleotide, σ is the concentration of the oligonucleotide after the reaction is performed for a period of t, and β is a constant.

In various embodiments, the incubation step is performed under isothermal conditions. The term “isothermal conditions” refers to a set of reaction conditions where the temperature of the reaction is kept essentially constant {i.e., at the same temperature or within the same narrow temperature range wherein the difference between an upper temperature and a lower temperature is no more than about 20° C.) during the course of the amplification. In certain embodiments, a reaction is carried out under conditions where the difference between an upper temperature and a lower temperature is no more than 15° C., 10° C., 5° C., 3° C., 2° C. or 1° C. Exemplary temperatures for isothermal amplification include, but are not limited to, any temperature between 50° C. to 70° C. or the temperature range between 50° C. to 70° C., 55° C. to 70° C., 60° C. to 70° C., 65° C. to 70° C., 50° C. to 55° C., 50° C. to 60° C., or 50° C. to 65° C.

In various embodiments, the isothermal conditions are a temperature of 57° C. and at least 60 amplification cycles re wherein each cycle is 55 seconds.

Such isothermal conditions mean that a set of reaction conditions where the temperature of the reaction is kept essentially constant during the course of the amplification. An advantage of the amplification method of the present invention is that there is no need to cycle the temperature between an upper temperature and a lower temperature. Both the nicking and the extension reaction will work at the same temperature or within the same narrow temperature range. However, it is not necessary that the temperature be maintained at precisely one temperature. If the equipment used to maintain an elevated temperature allows the temperature of the reaction mixture to vary by a few degrees this is not detrimental to the amplification reaction. For instance, both the nicking reaction using N.BstNB I (New England Biolabs) and the extension reaction using exo− Bst polymerases (BioRad) may be carried out at about 55° C. Other polymerases that are active between about 50° C. and 70° C. include, but are not limited to, exo− Vent (New England Biolabs), exo− Deep Vent (New England Biolabs), exo− Pfu (Strategene), exo-Bca (Panvera) and Sequencing Grade Taq (Promega). Restriction endonucleases that nick a hemimodified RERS and that are active between about 50° C. and 65° C. include, but are not limited to Bsr I, BstN I, BsmA I, Bsl I and BsoB I (New England BioLabs), and BstO I (Promega).

The extension/amplification reaction may be carried out in the presence of a labeled dideoxyribonucleoside triphosphate so that the label is incorporated into the amplified nucleic acid fragments. Labels suitable for incorporating into a nucleic acid fragment, and methods for the subsequent detection of the fragment are known in the art, and exemplary labels include, but are not limited to, a radiolabel such as 32P, 33p, 1251 or 35S, an enzyme capable of producing a colored reaction product such as alkaline phosphatase, fluorescent labels such as fluorescein isothiocyanate (FITC), biotin, avidin, digoxigenin, antigens, haptens or fluorochromes. The presence of the label in the amplified nucleic acid fragments allows these fragments to function as nucleic acid probes for detecting nucleic acids that are capable of hybridizing with the fragments.

In another aspect of the invention, there is provided a kit to amplify nucleic acid, the kit comprising:
(a) at least one primer for a target region on the first or second nucleic acid template;
(b) at least one protein having DNA polymerase domain function, wherein the domain function comprises a first domain function capable of strand displacement activity and a second domain function capable of high processivity activity, or at least one protein having DNA polymerase domain function capable of strand displacement activity and at least one protein having DNA polymerase domain function capable of high processivity activity, wherein the domain functions capable of strand displacement activity and high processivity activity are separate from each other and capable of carrying out their activities simultaneously;
(c) at least one deoxynucleoside triphosphate; and
(d) at least one nicking agent,
   and instructions for using the kit.

In various embodiments, the first and second domains are separated by a linker to form a chimeric protein.

In various embodiments, the protein having DNA polymerase domain function is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, T4 DNA polymerase, strand displacing Taq polymerase, and combinations thereof.

In various embodiments, the kit further comprises a reverse transcriptase.

The kits of the invention may further comprise a buffer for the nicking agent, a buffer for the DNA polymerase(s), or both buffers. Buffers may be included for suitable storage of such agents. Alternatively, the kits may further comprise a buffer suitable for both the nicking agent and the DNA polymerase. In some embodiment, the kits may also comprise a container containing a strand displacement facilitator, such as trehalose.

The instruction booklet provides information on how to use the kit of the present invention for amplifying nucleic acids without the required use of an external oligonucleotide primer. The information includes descriptions on how to use and/or store the nicking agent and the DNA polymerase, descriptions of buffer(s) for the nicking agent and the DNA polymerase, appropriate reaction temperature(s) and reaction time period(s), etc.

In order that the present invention may be fully understood and readily put into practical effect, there shall now be described by way of non-limitative examples only preferred embodiments of the present invention, the description being with reference to the accompanying illustrative figures.

### IN THE FIGURES

FIG. 1 shows an amplification according to the present invention (SENANG) compared with other known amplifications (e.g. EXPAR) for 21 bp long target oligonucleotides. It shows that SENANG can achieve 10⁶ fold or more within 10 minutes. Each “cycle” shown is 1 minute.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a method is disclosed and discussed and a number of modifications that can be made to the method steps are discussed, each and every combination and permutation of the method steps, and the modifications that are possible are specifically contemplated unless specifically indicated to the contrary. Likewise, any subset or combination of these is also specifically contemplated and disclosed. This concept applies to all aspects of this disclosure. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

This invention provides for a new class of isothermal amplification reaction method for amplifying DNA from RNA and DNA. These reactions synthesize specific oligonucleotides specified by a designed template. The target nucleotide can be amplified 10⁶ fold or more within 10 minutes.

The stochastic use of enzymes can be applied to many other enzymatic reactions not related to isothermal amplification.

This invention provides the following advantages:
1) The method of this invention is not limited to short nucleotides (8-16 bases).
2) The method is not limited to a target nucleotide sequence between 20 and 40 in length.
3) The target nucleotide can be amplified 10⁶ fold or more within 10 minutes.
4) Functional domains from multiple families of polymerase, endonuclease and reverse transcriptase are utilized for this amplification.

Advantageously, the amplification is extremely rapid and can be designed to operate at a range of temperatures. It is important to isolate some parts the reactions. Antibodies can be designed to block the action of respective proteins before the desired reaction temperature is reached.

In addition, as the reaction proceeds extremely rapidly, some commercial plate readers and fluorescence may be too slow to accurate compare the readouts between the first and the last well of a read. Faster readers will allow ‘real-time’ comparisons, and slower readers do so with a lower throughput (e.g. 8 wells instead of 96 or 384 wells). Nonetheless, the method is generic and can also be implemented with an end-point readout.

In addition, certain chemical compositions that allow unspecific amplification may be modified and used. ‘Novel proteins’ can be re-classified by splicing functional domains of various polymerases, reverse transcriptases and endonucleases. Also included in this application is the use of specific functional domains of DNA polymerases, instead of just focusing on the whole protein itself.

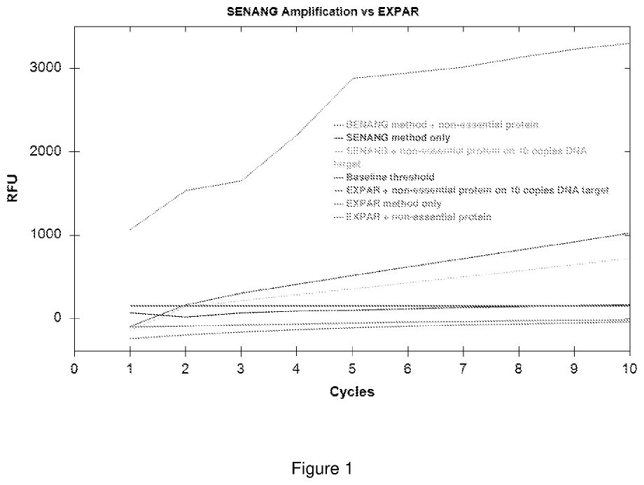

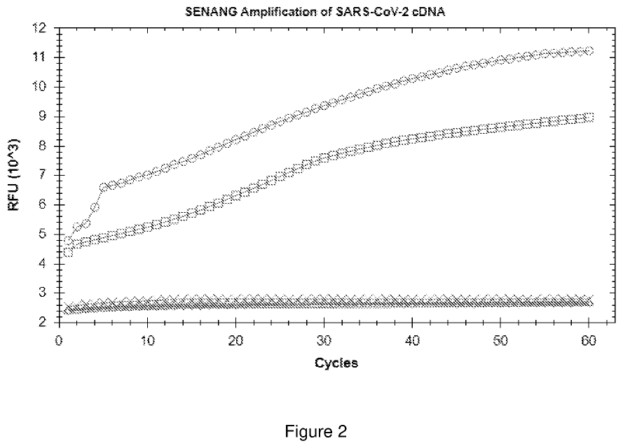

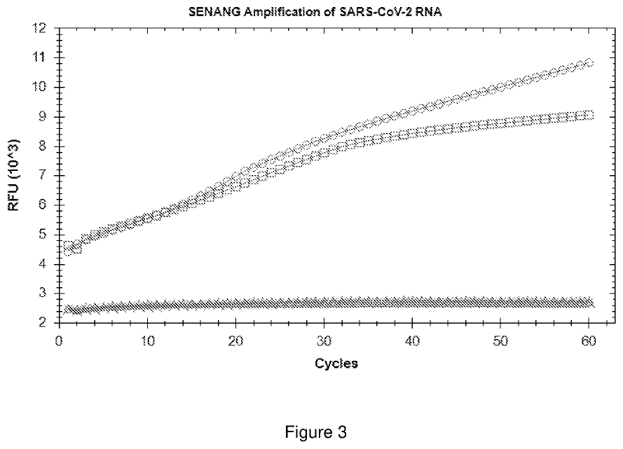

The present invention (also known as “SENANG”) is a new class of isothermal amplification reaction method for amplifying DNA from DNA and/or RNA that can yield 106 fold or more amplicons longer than 16 base pairs within 10 minutes (see FIG. 1). Currently, only two other isothermal amplification methods are able to match SENANG in speed: (1) Exponential Amplification Reaction (EXPAR) and (2) Nicking Enzyme Amplification Reaction (NEAR). However, both methods are limited to amplifying only an 8-16 base pair (bp) target sequence. NEAR's application, is limited, and is differentiated from EXPAR simply by adding another nicking enzyme recognition sequence to a conventional EXPAR template. As such, most of the focus of the comparison would be between SENANG and EXPAR.

Working Principle of SENANG

In general, both EXPAR and NEAR reactions require the use of a nicking enzyme, DNA polymerase with strand displacement activity and, optionally, a reverse transcriptase. The SENANG approach overcomes the 8-16 bp amplification limitation of EXPAR and NEAR by utilizing a protein with a DNA polymerase functional domain that is not strictly a polymerase with strand displacement activity. Specifically, a DNA polymerase domain without strand displacement activity can be used to overcome the 8-16 bp amplification limit by both conventional and engineered polymerases with strand displacement activity. This is done by a stochastic polymerization of DNA by a protein with a DNA polymerase domain that does not necessarily display strand displacement activity, unlike the requirements by EXPAR and NEAR.

Applications of SENANG

The applications in which SENANG can be applied to include all common applications of PCR and isothermal PCR, as well as library preparation for next-generation sequencing (NGS). SENANG's rapid amplification and increased limit of amplified oligonucleotide length additionally allows it to be used for applications beyond biotechnology, including for data storage processes. It is possible, for example, to use unique SENANG amplified oligo repeats to represent 64-bit information. This has exciting potential for data storage applications due to SENANG's relatively long, rapid and accurate ‘write’ mechanism.

Features

The method of the present invention employs the addition of at least one DNA polymerase functional domain to overcome the existing limitation of up to 16 base pairs amplification in the prior art (WO2004067726). Furthermore, the method is not limited to simply applying a second order repeat of the first exponential amplification cycle as detailed in WO2009012246A2.

Both documents WO2004067726 and WO2009012246 are incorporated by reference in their entireties.

### Example 1—Amplification cDNA of Sars-Cov-2 S Gene Using to a Method of the Invention

A portion of a synthetic cDNA of the S gene of SARS-CoV-2 was amplified at 57° C. over 60 cycles of 55 seconds each with the following amplification profile. There were 6 reaction set ups in this example and the amplification results are plotted and shown in FIG. 2.

The SENANG enhancer template in this reaction is a 48-mer oligonucleotide.

The 6 reactions in this setup can be assembled as follows:

Reaction 1

+NB.BsrDI+ Bst 3.0 Polymerase+Reverse Transcriptase+Pfu Polymerase (denoted by “circles” in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template to | 0.1 | μM |
| enable exponential amplification | | |
| 100× SYBR Green I | 5× | |
| SARS-CoV-2 cDNA Template | 1 | pM |
| SARS-CoV-2 RNA Template | 0 | |
| Pfu Polymerase | 2.5 | u |
| Taq Polymerase | 0 | |
| Reverse Transcriptase | 2.5 | u |

| | |
|---|---|
| Nuclease-free water | up to 10 μL |

Reaction 2

+NB.BsrDI+Bst 3.0 Polymerase+Reverse Transcriptase+Taq Polymerase (denoted by “triangles” in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 1 | pM |
| SARS-CoV-2 RNA Template | 0 | |
| Pfu Polymerase | 0 | |
| Taq Polymerase | 2.5 | u |
| Reverse Transcriptase | 2.5 | u |

| | |
|---|---|
| Nuclease-free water | up to 10 μL |

Reaction 3

+NB.BsrDI+Bst 3.0 Polymerase+Reverse Transcriptase (denoted by “crosses” in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 1 | pM |
| SARS-CoV-2 RNA Template | 0 | |
| Pfu Polymerase | 0 | |
| Taq Polymerase | 0 | |
| Reverse Transcriptase | 2.5 | u |

| | |
|---|---|
| Nuclease-free water | up to 10 μL |

Reaction 4

+NB.BsrDI+Bst 3.0 Polymerase+Pfu Polymerase (denoted by “squares” in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 1 | pM |
| SARS-CoV-2 RNA Template | 0 | |
| Pfu Polymerase | 2.5 | u |

| | |
|---|---|
| Taq Polymerase | 0 |
| Reverse Transcriptase | 0 |
| Nuclease-free water | up to 10 μL |

Reaction 5

+NB.BsrDI+Bst 3.0 Polymerase+Taq Polymerase (denoted by “diamonds” in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 1 | pM |
| SARS-CoV-2 RNA Template | 0 | |
| Pfu Polymerase | 0 | |
| Taq Polymerase | 2.5 | u |

| | |
|---|---|
| Reverse Transcriptase | 0 |
| Nuclease-free water | up to 10 μL |

Reaction 6

+NB.BsrDI+Bst 3.0 Polymerase (line with no denotations in FIG. 2)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 1 | pM |

| | |
|---|---|
| SARS-CoV-2 RNA Template | 0 |
| Pfu Polymerase | 0 |
| Taq Polymerase | 0 |
| Reverse Transcriptase | 0 |
| Nuclease-free water | up to 10 μL |

In this example, the following enhancer template sequence and primer sequence are as follows:

SENANG enhancer template sequence:

GCACCAAGTGACATAGTGTAGCATTGCGCACCAAGTGACATAGTGTAG
Forward primer:
CACGTAGTGTAGCTAGTCAATCCAT
Reverse primer:
TCTGCACCAAGTGACATAGTGTAG

SARS-CoV-2 cDNA Template:

cDNA template is in vitro transcribed from RNA synthesized based on the sequence of Genbank ID: MN908947.3 of the SARS-CoV-2 Genome

SARS-CoV-2 RNA Template:

RNA template is synthetically synthesized based on the sequence of Genbank ID: MN908947.3 of the SARS-CoV-2 Genome

Based on the results shown in FIG. 2, the experiments show that Reaction 1 provides the best amplification results.

### Example 2: Amplification of RNA of Sars-Cov-2 S Gene Using the Method of the Invention

A portion of in vitro transcribed RNA of the S gene of SARS-CoV-2 was amplified at 57° C. over 60 cycles of 55 seconds each with the following amplification profile. There were 6 reaction set ups in this example and the amplification results are plotted and shown in FIG. 3.

The SENANG enhancer template in this reaction is a 48-mer oligonucleotide.

The 6 reactions in this setup can be assembled as follows:

Reaction 1

+NB.BsrDL+Bst 3.0 Polymerase+Reverse Transcriptase+Pfu Polymerase (denoted by “circles” in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |
| Pfu Polymerase | 2.5 | u |
| Taq Polymerase | 0 | |
| Reverse Transcriptase | 2.5 | u |

| | |
|---|---|
| Nuclease-free water | up to 10 μL |

Reaction 2

+NB.BsrDI+Bst 3.0 Polymerase+Reverse Transcriptase+Taq Polymerase (denoted by “triangles” in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |
| Pfu Polymerase | 0 | |
| Taq Polymerase | 2.5 | u |
| Reverse Transcriptase | 2.5 | u |

| | |
|---|---|
| Nuclease-free water | up to 10 μL |

Reaction 3

+NB.BsrDI+Bst 3.0 Polymerase+Reverse Transcriptase (denoted by “crosses” in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |
| Pfu Polymerase | 0 | |
| Taq Polymerase | 0 | |
| Reverse Transcriptase | 2.5 | u |

| | |
|---|---|
| Nuclease-free water | up to 10 μL |

Reaction 4

+NB.BsrDI+Bst 3.0 Polymerase+Pfu Polymerase (denoted by “squares” in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |
| Pfu Polymerase | 2.5 | u |

| | |
|---|---|
| Taq Polymerase | 0 |
| Reverse Transcriptase | 0 |
| Nuclease-free water | up to 10 μL |

Reaction 5

+NB.BsrDI+Bst 3.0 Polymerase+Taq Polymerase (denoted by “diamonds” in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |
| Pfu Polymerase | 0 | |
| Taq Polymerase | 2.5 | u |

| | |
|---|---|
| Reverse Transcriptase | 0 |
| Nuclease-free water | up to 10 μL |

Reaction 6

+NB.BsrDI+Bst 3.0 Polymerase (line with no denotations in FIG. 3)

| dNTPs | 0.5 | mM |
|---|---|---|
| Forward Primer | 50 | nM |
| Reverse Primer | 50 | nM |
| MgCl₂ | 2.5 | mM |
| Promega GoScript Reaction Buffer | 1× | |
| NEB NB. BsrDI | 0.4 | u/μL |
| NEB Bst 3.0 Polymerase | 0.05 | u/μL |
| SENANG enhancer template | 0.1 | μM |
| 100× SYBR Green I | 5× | |
| SARS-CoV-2 cDNA Template | 0 | |
| SARS-CoV-2 RNA Template | 1 | pM |

| | |
|---|---|
| Pfu Polymerase | 0 |
| Taq Polymerase | 0 |
| Reverse Transcriptase | 0 |
| Nuclease-free water | up to 10 μL |

In this example, the following enhancer template sequence and primer sequence are as follows:

SENANG enhancer template sequence:

GCACCAAGTGACATAGTGTAGCATTGCGCACCAAGTGACATAGTGTAG
Forward primer:
CACGTAGTGTAGCTAGTCAATCCAT
Reverse primer:
TCTGCACCAAGTGACATAGTGTAG

SARS-CoV-2 cDNA Template:

cDNA template is in vitro transcribed from RNA synthesized based on the sequence of Genbank ID: MN908947.3 of the SARS-CoV-2 Genome

SARS-CoV-2 RNA Template:

RNA template is synthetically synthesized based on the sequence of Genbank ID: MN908947.3 of the SARS-CoV-2 Genome

Based on the results shown in FIG. 2, the experiments show that Reaction 1 provides the best amplification results.

Whilst there has been described in the foregoing description preferred embodiments of the present invention, it will be understood by those skilled in the technology concerned that many variations or modifications in details of design or construction may be made without departing from the present invention.

1. A method to amplify nucleic acid, the method comprising:
(a) forming a reaction mixture comprising:
(i) a first nucleic acid template comprising a strand having a first nicking agent recognition sequence;
(ii) a second nucleic acid template comprising a strand having a second nicking agent recognition sequence;
(iii) at least one primer for a target region on the first or second nucleic acid template;
(iv) at least one protein having DNA polymerase domain function, wherein the domain function comprises a first domain function capable of strand displacement activity and a second domain function capable of high processivity activity, or
at least one protein having DNA polymerase domain function capable of strand displacement activity and at least one protein having DNA polymerase domain function capable of high processivity activity;
(v) at least one deoxynucleoside triphosphate; and
(vi) a first nicking agent for recognizing the first nicking agent recognition sequence and a second nicking agent for recognizing the second nicking agent recognition sequence;
(b) incubating the reaction mixture under conditions that amplifies the nucleic acid templates,
wherein the domain functions capable of strand displacement activity and high processivity activity are separate from each other and capable of carrying out their activities simultaneously.

2. The method according to claim 1, wherein the first and second domains are separated by a linker to form a chimeric protein.

3. The method according to any one of the preceding claims, wherein the protein having DNA polymerase domain function is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, T4 DNA polymerase, strand displacing Taq polymerase, and combinations thereof.

4. The method according to any one of the preceding claims, wherein the second template further comprises a region that is not substantially complementary to the at least one primer, and incubating the reaction mixture under conditions to amplify:
(a) a first single-stranded nucleic acid molecule using the first template nucleic acid as a template; and
(b) a second single-stranded nucleic acid molecule using the second template nucleic acid as a template.

5. The method according to any one of the preceding claims, wherein the reaction mixture further comprises a third nucleic acid template comprising:
(a) a third nicking agent recognition sequence;
(b) a first region that is substantially identical or complementary to the at least one primer; and
(c) a second region that is not substantially identical or complementary to the at least one primer.

6. The method according to claim 5, wherein the first, second and third nicking agent recognition sequences are identical.

7. The method according to any one of the preceding claims, wherein the nicking agent is a nicking endonuclease selected from the group consisting of Nb.BbvCI, Nb.Bsml, Nb.BsrDI, Nb.BssSI, Nb.Btsl, Nt.Alwl, Nt.BbvCI, Nt.BsmAl, Nt.BspQI, Nt.BstNBl, Nt.CviPII, Zinc Finger nickase, TALE nickase, Cas nickase, and combinations thereof.

8. The method according to any one of the preceding claims, wherein the 3′ terminus of the first nucleic acid template or the second nucleic acid template is blocked.

9. The method according to any one of the preceding claims, wherein first nucleic acid template and the second nucleic acid template are immobilized.

10. The method according to any one of the preceding claims, wherein first nucleic acid template or the second nucleic acid template comprises nucleic acid modifications.

11. The method according to any one of the preceding claims, wherein first nucleic acid template or the second nucleic acid template is about 6 to 20,000 nucleotides in length.

12. The method according to any one of the preceding claims, further comprising the step of characterizing the amplified nucleic acid.

13. The method according to claim 12, wherein the characterizing step is performed by a technique selected from the group consisting of luminescence spectroscopy or spectrometry, fluorescence spectroscopy or spectrometry, mass spectrometry, liquid chromatography, fluorescence polarization, electrophoresis, mass spectrometry, SYBR I fluorescence, SYBR II fluorescence, SYBR Gold, Pico Green, Evagreen, TOTO-3, intercalating dye detection, FRET, molecular beacon detection, scorpion probe detection, surface capture, capillary electrophoresis, incorporation of labeled nucleotides to allow detection by capture, fluorescence polarization, lateral flow capture, and combinations thereof.

14. The method according to any one of the preceding claims, wherein the reaction mixture further comprises a template conjugate for linking the first and second nucleic acid templates.

15. The method according to claim 14, wherein the 3′ terminus of the first oligonucleotide template is linked to the 5′ terminus of the second oligonucleotide template via the conjugate.

16. The method according to claim 14, wherein the 3′ terminus of the first oligonucleotide template is linked to the 3′ terminus of the second oligonucleotide template via the conjugate.

17. The method according to any one of the preceding claims, wherein the first nucleic acid template is identical to the second nucleic acid template.

18. The method according to any one of the preceding claims, wherein the first nicking agent recognition sequence is identical to the second nicking agent recognition sequence.

19. The method according to any one of the preceding claims, wherein the nucleic acid template further comprises a detectable label.

20. The method according to claim 19, wherein the detectable label is a fluorescent moiety.

21. The method according to any one of the preceding claims, wherein the amplification has the kinetic that fits the equation σ˜σ₀ e^{βt}, where σ₀ is an initial concentration of the oligonucleotide, σ is the concentration of the oligonucleotide after the reaction is performed for a period of t, and β is a constant.

22. The method according to any one of the preceding claims, wherein the incubation step is performed under isothermal conditions.

23. A kit to amplify nucleic acid, the kit comprising:
(a) at least one primer for a target region on the first or second nucleic acid template;
(b) at least one protein having DNA polymerase domain function, wherein the domain function comprises a first domain function capable of strand displacement activity and a second domain function capable of high processivity activity, or
at least one protein having DNA polymerase domain function capable of strand displacement activity and at least one protein having DNA polymerase domain function capable of high processivity activity, wherein the domain functions capable of strand displacement activity and high processivity activity are separate from each other and capable of carrying out their activities simultaneously;
(c) at least one deoxynucleoside triphosphate; and
(d) at least one nicking agent,
and instructions for using the kit.

24. The kit according to claim 23, wherein the first and second domains are separated by a linker to form a chimeric protein.

25. The kit according to any one of claim 23 or 24, wherein the protein having DNA polymerase domain function is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, T4 DNA polymerase, strand displacing Taq polymerase, and combinations thereof.

26. The kit according to any one of claims 23 to 25, further comprising a reverse transcriptase.
